(19) United States
(12) Patent Application Publication
(10) Pub. No.: US 2023/0073240 A1
(43) Pub. Date: Mar. 9, 2023

(54) AIR PURIFIER

(71) Applicant: SHENZHEN ANTOP TECHNOLOGY CO., LTD., Shenzhen (CN)

(72) Inventors: Ruidian YANG, Shenzhen (CN); Chu YAN, Shenzhen (CN)

(21) Appl. No.: 17/532,882

(22) Filed: Nov. 22, 2021

(30) Foreign Application Priority Data
Sep. 6, 2021 (CN) 202122142916.9

(51) Int. Cl. B01D 46/00; B01D 46/24; B01D 53/00; B01D 53/86; A61L 9/20

(52) U.S. Cl. CPC B01D 46/0028; B01D 46/2403; B01D 53/007; B01D 46/0049; B01D 53/8671; A61L 9/205; B01D 2265/029; B01D 2265/028; B01D 2273/30; B01D 2255/2073; B01D 2255/802; B01D 2255/9155; B01D 2257/708; B01D 2257/7027; B01D 2259/804; B01D 2279/65; A61L 2209/14

(57) ABSTRACT

This application provides an air purifier, which includes a casing, a fan, a filter, and a purification module. The filter is in a cylindrical shape. The purification module includes an inner filter for purification of harmful gases in air. The inner filter includes an inner filter cylinder and a purification plate, the purification plate and/or the inner filter cylinder are honeycomb-like or reticulate, and the inner filter cylinder is disposed in the filter. In the present application, the air purifier is provided with an inner filter to remove harmful gases such as formaldehyde in the air, and the purification plate and/or inner filter cylinder are honeycomb-like or reticulate to reduce wind resistance of the inner filter and reduce air resistance, thereby reducing the fan speed and noise.

### CROSS-REFERENCE TO RELATED APPLICATION

Pursuant to 35 U.S.C. § 119 and the Paris Convention, this application claims the benefit of Chinese Patent Application No. 202122142916.9 filed on Sep. 6, 2021, the contents of which are incorporated herein by reference thereto.

### TECHNICAL FIELD

The present application relates to the field of air purification technology, and more particularly to an air purifier.

### BACKGROUND

As people pay more attention to the environment, the use of air purifiers is increasing. An air purifier generally draws air through a fan to generate suction, so that the air is filtered and purified by a filter assembly, and then sent out from an air outlet. For a vertical air purifier, filter cartridge is generally used to suck air around the filter cartridge to improve purification efficiency. In order to improve the purification effect, especially to remove harmful gases such as formaldehyde in the air, materials such as filter paper and activated carbon filter elements are generally used, where these materials are stacked, and then folded to form a filter cartridge. Such that the harmful gases in the air can be removed as the air passes through the filter cartridge. However, this structure has a large wind resistance, which requires the fan to generate greater suction during purification, and the noise of the fan increases, which results in a louder noise from the air purifier.

### SUMMARY

An object of embodiments in the present application is to provide an air purifier to solve the problem that the air purifier in the existing technologies uses a filter cartridge that removes harmful gases such as formaldehyde, but has large wind resistance and high fan operating noise.

To achieve the above object, the technical solution adopted in the embodiments of the present application is to provide an air purifier, including a casing, a fan, and a filter, the fan and the filter are installed in the casing. The casing defines air intake holes at a position in the proximity of the filter, and defines an air outlet at an end of the casing in the proximity of an air outlet end of the fan. The filter is in a cylindrical shape. The air purifier further includes a purification module which includes an inner filter for purification of harmful gases in air. The inner filter includes an inner filter cylinder arranged in a cylindrical shape and a purification plate for the purification of harmful gases in the air. The purification plate is installed in the inner filter cylinder. The purification plate is honeycomb-like or reticulate. The inner filter cylinder is disposed in the filter, and an upper end of the inner filter cylinder is supported in the casing.

In an optional embodiment, the upper end of the inner filter cylinder is adaptively connected to an upper end of the filter.

In an optional embodiment, the upper end of the filter is provided with an internal thread, the upper end of the inner filter cylinder is provided with an external thread, and the internal thread is engaged with the external thread in a fitted manner; or the upper end of the filter is provided with an inner rotary buckle, and the upper end of the inner filter cylinder is provided with an outer rotary buckle, and the inner rotary buckle is engaged in a snap-fitted manner with the outer rotary buckle.

In an optional embodiment, the inner filter cylinder is a photocatalytic filter, and the purification module further includes a light source installed in the casing and configured to irradiate the inner filter cylinder.

In an optional embodiment, the purification module further includes a light source installed in the casing and configured for emitting ultraviolet light toward the inner filter cylinder.

In an optional embodiment, the purification plate is made of a honeycomb-like or reticulate substrate. The substrate is an activated carbon cardboard; or the substrate is a honeycomb-like carbon-filled filter; or the substrate has a surface being sprayed with a coating that removes harmful gases in the air, and the coating is a manganese compound layer, a photocatalyst layer or a cold catalyst layer.

In an optional embodiment, the inner filter cylinder is a honeycomb-like cylinder or a reticulate cylinder.

In an optional embodiment, the inner filter cylinder is made of a honeycomb-like or reticulate substrate. The substrate is an activated carbon cardboard; or the substrate is a honeycomb-like carbon-filled filter; or the substrate has a surface being sprayed with a coating that removes harmful gases in the air, wherein the coating is a manganese compound layer, a photocatalyst layer or a cold catalyst layer.

The beneficial effects of the air purifier provided by the embodiments of the present application are that: compared with the prior art, the air purifier of the present application has a purification plate provided in the inner filter cylinder to achieve an effect of removing harmful gases in the air. The purification plate is honeycomb-like or reticulate to reduce the wind resistance of the inner filter and reduce the air resistance, thereby reducing the speed and noise of the fan. The filter is in a cylindrical shape, and the inner filter cylinder is disposed in the filter, which reduces occupied space, and the air filtered by the filter can better enter the inner filter, and then enter the fan, thus the purification efficiency is higher.

Another object of the embodiments in the present application is to provide an air purifier, including a casing, a fan, and a filter. The fan and the filter are installed in the casing. The casing defines air intake holes at a position in the proximity of the filter, and defines an air outlet at an end of the casing in the proximity of an air outlet end of the fan. The filter is in a cylindrical shape. The air purifier further includes a purification module which includes an inner filter for purification of harmful gases in air. The inner filter includes an inner filter cylinder for the purification of harmful gases in the air and a purification plate for purifying the air. The purification plate is installed at a bottom of the inner filter cylinder. The inner filter cylinder is a honeycomb-like cylinder or a reticulate cylinder. The inner filter cylinder is disposed in the filter, and an upper end of the inner filter cylinder is supported in the casing.

In an optional embodiment, the inner filter cylinder is made of a honeycomb-like or reticulate substrate. The substrate is an activated carbon cardboard; or the substrate is a honeycomb-like carbon-filled filter; or the substrate has a surface being sprayed with a coating that removes harmful gases in the air, and the coating is a manganese compound layer, a photocatalyst layer or a cold catalyst layer.

The beneficial effects of the air purifier provided by the embodiments of the present application are that: compared with the prior art, the air purifier of the present application is provided with an inner filter cylinder that can remove harmful gases in the air to improve the purification capability. The inner filter cylinder is a honeycomb-like cylinder or a reticulate cylinder to reduce the wind resistance of the inner filter and reduce the air resistance, thereby reducing the speed and noise of the fan. The filter is in a cylindrical shape, and the inner filter cylinder is disposed in the filter, which reduces occupied space, and the air filtered by the filter can better enter the inner filter, and then enter the fan, thus the purification efficiency is higher.

### BRIEF DESCRIPTION OF THE DRAWINGS

In order to illustrate the technical solutions in the embodiments of the present application more clearly, the drawings that need to be used in the description of the embodiments or exemplary technologies will be briefly described herein below. Obviously, the drawings in the following description are merely some embodiments of the present application, for those of ordinary skill in the art can obtain other drawings on the basis of these drawings without creative labor.

FIG. 1 is a schematic front view of a structure of an air purifier provided by an embodiment of the present application;

FIG. 2 is a schematic cross-sectional structure diagram of the air purifier taken along the line A-A in FIG. 1;

FIG. 3 is a schematic diagram of an explosive structure of the air purifier shown in FIG. 1;

FIG. 4 is a schematic structural diagram of a filter in FIG. 2 when it is separated from the inner filter cylinder;

FIG. 5 is a schematic structural diagram of the separated filter of the air purifier shown in FIG. 1;

FIG. 6 is an enlarged view of part A in FIG. 5;

FIG. 7 is an enlarged view of part B in FIG. 5;

FIG. 8 is a schematic structural diagram of an upper casing of the air purifier shown in FIG. 2;

FIG. 9 is a schematic structural diagram of a fan in the air purifier shown in FIG. 2;

FIG. 10 is a schematic sectional view of the fan in the air purifier shown in FIG. 9;

FIG. 11 is a schematic diagram of an explosive structure of the fan in the air purifier shown in FIG. 9;

FIG. 12 is a schematic structural diagram of a diffuser part of the fan shown in FIG. 9;

FIG. 13 is a schematic cross-sectional structure diagram of an air purifier provided by another embodiment of the present application; and

FIG. 14 is a schematic cross-sectional structure diagram of an air purifier provided by another embodiment of the present application.

Among them, the main reference signs in the figures are listed as follows:
100—air purifier;
10—casing; 11—upper casing; 110—air outlet holes; 111—rib; 112—connection ring; 12—lower casing; 121—air intake holes; 122—reinforcement ring; 123—support rib; 1231—positioning groove; 124—groove;
20—fan; 21—housing; 210—air inlet; 211—support plate; 212—shrinking section; 2121—first shrinking portion; 2122—second shrinking portion; 213—convex ring; 214—air guide grid; 215—support bar; 216—fixing plate; 22—wind rotor; 221—moving blades; 222—baffle; 2221—flat plate portion; 2222—inclined portion; 223—intake guide ring; 2231—convex edge; 23—motor 231—output shaft; 24—diffuser; 241—inner ring plate; 242—outer ring plate; 243—stator blades; 25—mounting plate; 251—flat plate portion; 252—inclined portion; 253—through hole; 26—cover plate; 201—cavity;
30—filter; 31—filter cylinder; 32—bottom cover; 321—convex rib; 322—positioning convex; 323—limit protrusion; 324—recess; 325—knob; 33—ring cover; 331—inner rotary buckle;
40—purification module; 41—LED module; 42—inner filter; 421—inner filter cylinder; 4211—vents; 422—purification plate; 4221—openings; 423—outer rotary buckle;
51—panel; 52—support shell; 53—air guide shell; 531—annular section; 532—contraction section; 533—support plate;
61—negative ion generator; 62—negative ion emission needle.

### DETAILED DESCRIPTION OF THE EMBODIMENTS

In order to make the objects, technical solutions and advantages of the present application more comprehensible, the present application will be further described in detail below with reference to the accompanying drawings and specific embodiments. It should be understood that the specific embodiments described here are merely used to illustrate the present application, and are not intended to limit the present application.

It should be noted that when an element is referred to as being “fixed to” or “disposed/provided on” another element, it can be directly on the other element or indirectly on the other element. When an element is referred to as being “connected to” another element, it can be directly connected to the other element or indirectly connected to the other element.

In addition, the terms “first” and “second” are only used for descriptive purposes, and cannot be understood as indicating or implying relative importance or implicitly indicating the number of indicated technical features. Thus, the features defined with “first” and “second” may explicitly or implicitly include one or more of these features. In the description of the present application, “a/the plurality of” means two or more, unless otherwise specifically defined.

In the description of the present application, it should be understood that direction or position relationship indicated by terms of “center,” “longitudinal,” “transverse,” “length,” “width,” “thickness,” “upper,” “lower,” “front,” “back,” “left,” “right,” “vertical,” “horizontal,” “top,” “bottom,” “inner,” “outer” and the like, are based on the orientation or position relationship shown in the drawings, which are merely used for the convenience of describing the present application and simplifying the description, rather than indicating or implying that the device or element referred to must have a specific orientation, be constructed and operated in a specific orientation, it thus cannot be understood as a limitation to the present application.

In the present application, it should be noted that, unless otherwise clearly specified and defined, the terms “installed/mounted,” “in connection with,” “connected/coupled,” “fixed” should be understood in a broad sense. For example, they may be connected or detachably connected or integrated; they may be connected in a mechanical connection or an electrical connection; they may be directly connected or indirectly connected through an intermediate medium, and it may be an internal communication of two elements or an interaction relationship between two elements. For those of ordinary skill in the art, the specific meaning of the above-mentioned terms in the present application can be understood according to specific circumstances.

In the present application, description with reference to terms “an/one embodiment,” “some embodiments” or “the embodiment” means specific features, structure, or characteristics described in conjunction with the embodiment may be included in one or more embodiment of the present application. Therefore, the terms “in one embodiment,” “in some embodiments,” “in some other embodiments,” “in some other embodiments” etc. used in different places in this specification are not necessarily all refer to the same embodiment, instead, it means one or more but not all embodiments, unless otherwise specifically emphasized in other ways. In addition, the specific features, structures, or characteristics in one or more embodiments may be combined in any suitable manner.

The English original expression corresponding to the English abbreviations used in this application are as follows:

LED: Light Emitting Diode.

TVOC: Total Volatile Organic Compounds.

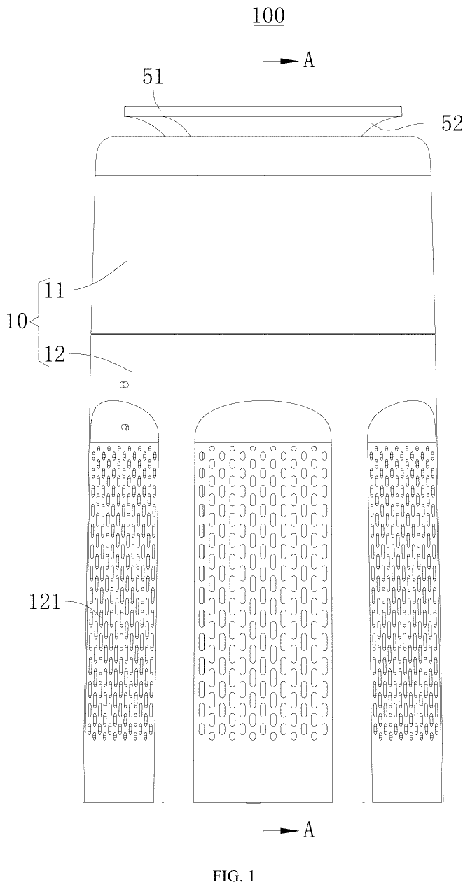

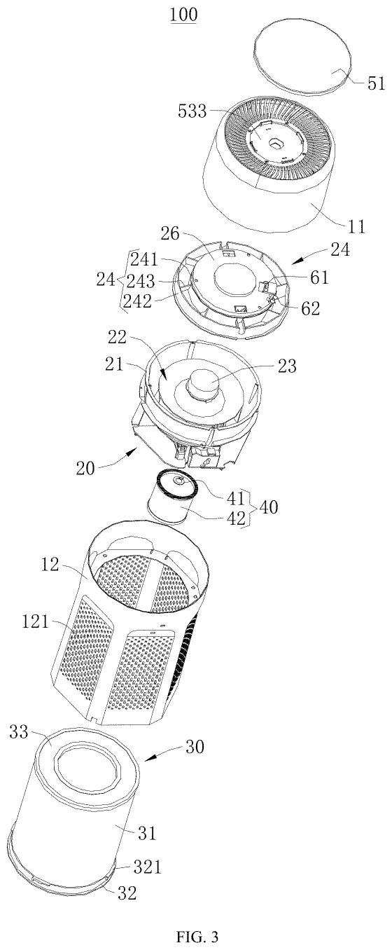

Referring to FIGS. 1 to 3, an air purifier 100 provided in the present application is described hereinbelow. The air purifier 100 includes: a casing 10, a fan 20, a filter 30, and a purification module 40. The fan 20, the filter 30 and the purification module 40 are respectively installed in the casing 10, and the fan 20, filter 30 and purification module 40 are supported and protected by the casing 10.

When the fan 20 is running, along the air flow direction, an end where the air enters the fan 20 is an air inlet end of the fan 20, and an end where the air flows out of the fan 20 is an air outlet end of the fan 20.

The casing 10 is provided with air intake holes 121 disposed at a position corresponding to the filter 30. The casing 10 is also provided with an air outlet 110 disposed at an end of the casing 10 close to an air outlet end of the fan 20.

The purification module 40 is provided between the filter 30 and the fan 20. The purification module 40 can decompose organic molecules in the passing air to remove harmful gases in the air, such as formaldehyde, benzene, TVOC and the like, and can play a sterilization effect to improve the purification performance.

During use, as the fan 20 runs, air enters the casing 10 from the air intake holes 121, thereby being filtered by the filter 30, purified by the purification module 40, and then enters the fan 20 through the air inlet end of the fan 20 to be pressurized, and then flows out from the air outlet end of the fan 20, and then flows out of the casing 10 through the air outlet 110 to achieve air purification.

The filter 30 is in a cylindrical shape. The purification module 40 includes an inner filter 42, which includes an inner filter cylinder 421 and a purification plate 422. The inner filter cylinder 421 is in a cylindrical shape, and the purification plate 422 is installed in the inner filter cylinder 421. The purification plate 422 is configured to remove harmful gases in the air, such as formaldehyde, benzene, TVOC and the like. The inner filter cylinder 421 is disposed in the filter 30 to reduce the occupied space and improve the space utilization rate. The inner filter cylinder 421 is supported in the casing 10, thereby the inner filter 42 can be supported in the casing 10.

The purification plate 422 may be honeycomb-like or reticulate. The purification plate 422 is honeycomb-like, that is, the purification plate 422 is provided with a plurality of honeycomb-like openings 4221. The use of the honeycomb-like purification plate 422 provides low resistance to airflow, thus more airflow can pass through the purification plate to improve the ability to remove formaldehyde, benzene, TVOC and other harmful gases. The inner filter cylinder 421 can also play a role in filtering air, improving the air purification capability. The purification plate 422 is reticulate, that is, the purification plate 422 is provided with a plurality of openings 4221 so that the purification plate 422 is reticulate. The use of the reticulate purification plate 422 provides low resistance to airflow, thus more airflow can pass through the purification plate to improve the ability to remove formaldehyde, benzene, TVOC and other harmful gases.

Compared with the existing technologies, the air purifier 100 provided by the present application is provided with a purification plate 422 in the inner filter cylinder 421 to achieve the effect of removing harmful gases in the air. The purification plate 422 may be honeycomb-like or reticulate so as to reduce wind resistance of the inner filter 42 and reduce air resistance, thereby reducing the speed and noise of the fan 20. The filter 30 is in a cylindrical shape, and the inner filter cylinder 421 is disposed in the filter 30, the occupied space can be reduced, and the air filtered by the filter 30 can be better filtered in the inner filter 42, and then enter the fan 20, so that the purification efficiency is higher.

In one embodiment, the purification plate 422 is provided at a bottom of the inner filter cylinder 421 to facilitate assembly. In other embodiments, the purification plate 422 may also be provided inside the inner filter cylinder 421, that is, the inner filter cylinder 421 is arranged around the purification plate 422, for example, the purification plate 422 may be arranged at the middle or upper part of the inner filter cylinder 421 in the height direction.

In one embodiment, an upper end of the inner filter cylinder 421 can be connected to an upper end of a filter cylinder 31, such that the inner filter cylinder 421 can be taken out of the casing 10 during a disassembling of the filter 30. In this way, when replacing the filter 30, the inner filter cylinder 421 can be replaced at the same time. It should be noted that the inner filter cylinder 421 can also be cleaned during a replacement of the filter 30.

In one embodiment, the upper end of the inner filter cylinder 421 is matched with the upper end of the filter 30, so that an outer side of the inner filter cylinder 421 is adaptively connected to an inner side of the filter 30, so that a gap between the inner filter cylinder 421 and the filter 30 can be small, so that an inner diameter of the inner filter cylinder 421 can be made larger, thereby an area of the inner filter cylinder 421 can be increased to improve the purification capability and allow more air to pass through the purification plate 422 to improve the air purification capability.

In one embodiment, the inner filter cylinder 421 is a photocatalytic filter, that is to say, the inner filter cylinder 421 is made of a photocatalytic net, or the inner filter cylinder 421 is provided with a photocatalytic net. The purification module 40 also includes a light source, which is installed in and supported by the casing 10. The light source is configured to irradiate the inner filter cylinder 421 so that the inner filter cylinder 421 can decompose organic molecules in the passing air such as harmful molecules of formaldehyde, benzene, TVOC, so as to play a role in purification and sterilization, which improves purification capability. It should be understood that the inner filter cylinder 421 may also use a high-efficiency filter layer to improve the air purification capability.

In an embodiment, the purification module 40 also includes a light source, which may be an ultraviolet light source. The light source is installed in the casing 10 to be supported by the casing 10. The light source emits ultraviolet light to the inner filter cylinder 421 so as to perform sterilization and disinfection.

In one embodiment, the purification plate 422 may be made of a substrate, and the substrate is honeycomb-like or reticulate, so that the purification plate 422 made by this substrate is honeycomb-like or reticulate.

In one embodiment, the substrate may be made of activated carbon corrugated paper to form a reticulate structure so as to remove harmful gases such as formaldehyde, benzene, TVOC and the like in the air.

In one embodiment, the substrate may be made of a honeycomb-like activated carbon to form a honeycomb-like or reticulate structure so as to remove harmful gases such as formaldehyde, benzene, TVOC, etc. in the air.

In one embodiment, the substrate can be made of a honeycomb-like carbon-filled filter screen, that is, a honeycomb-like support plate is disposed between two layers of filter screens, and activated carbon particles are disposed in the holes of the honeycomb-like support plate to form a honeycomb-like or reticulate structure, so that harmful gases such as formaldehyde, benzene, TVOC, etc. can be removed from the air.

In one embodiment, the substrate may be made of a honeycomb-like or reticulate substrate with a coating being sprayed on the surface that removes harmful gases in the air. The coating may be a manganese compound layer, a photocatalyst layer or a cold catalyst layer, etc., so that the substrate can remove harmful gases such as formaldehyde, benzene, and TVOC in the air.

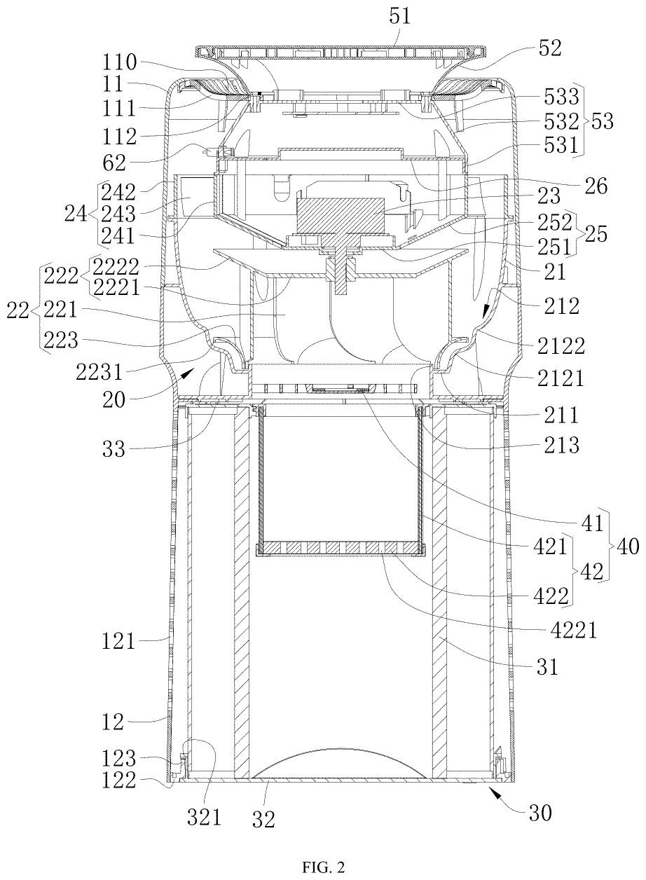

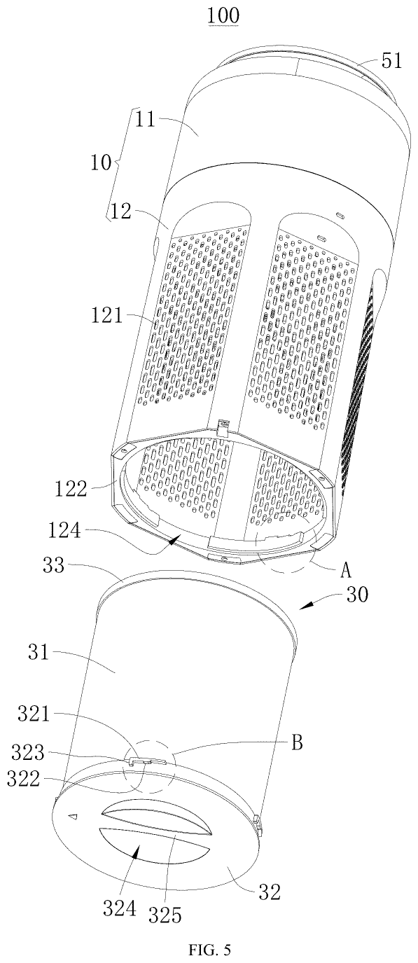

In one embodiment, referring to FIGS. 2 and 5, the casing 10 has an open bottom, and the filter 30 includes the filter cylinder 31 and a bottom cover 32. The bottom cover 32 is mounted on a bottom of the filter cylinder 31 to form a cylindrical filter 30. The inner filter cylinder 421 is arranged in the filter cylinder 31. During assembling, the filter cylinder 31 is extended into the casing 10, the bottom cover 32 is detachably covered on the bottom of the casing 10, and the side of the casing 10 is provided with a plurality of air intake holes 121 corresponding to the position of the filter cylinder 31. The bottom of the casing 10 is set to be open, and the bottom cover 32 of the filter 30 is covered on the bottom of the casing 10, so that the bottom cover 32 of the filter 30 is used as the bottom cover 32 of the casing 10. When the filter 30 needs to be replaced, the bottom cover 32 will be replaced together, that is to say, the bottom cover 32 and the filter cylinder 31 form an integrated structure as a consumable, so that there is no need to worry about the wear and aging of the bottom cover 32, and thus the service life of the air purifier 100 is greatly increased.

In one embodiment, the bottom cover 32 and the filter cylinder 31 are fixed into an integral structure, so that when the filter 30 is replaced, it is more convenient to disassemble and assemble.

In one embodiment, referring to FIGS. 1 to 3, when the casing 10 includes an upper casing 11 and a lower casing 12, the air intake holes 121 are provided on a side of the lower casing 12, and the lower casing 12 has an open bottom, and the air outlet 110 is provided on the upper casing 11.

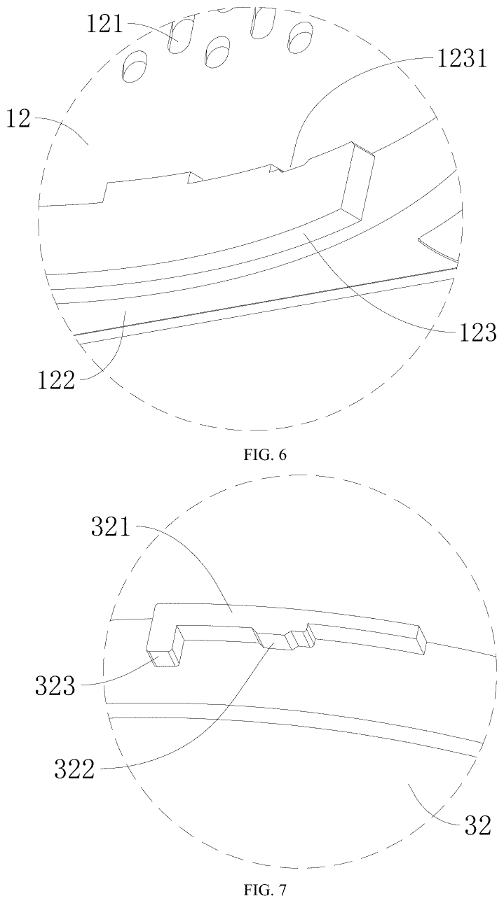

In one embodiment, referring to FIGS. 5, 6 and 7, an inner surface of the lower end of the casing 10 is provided with a reinforcement ring 122 to increase the structural strength of the lower end of the casing 10, so as to facilitate the installation and fixing of the bottom cover 32 of the filter 30.

In one embodiment, the inner surface of the lower end of the casing 10 is provided with a plurality of support ribs 123. A side surface of the bottom cover 32 is provided with a plurality of convex ribs 321, and the convex rib 321 is configure to be supported on the support rib 123, namely, the bottom cover 32 is mounted on the casing 10 by fitting the convex rib 321 on the support rib 123. Between two adjacent support ribs 123, a groove 124 is provided for the convex rib 321 to pass through. That is to say, during assembly, the convex rib 321 is inserted into the casing 10 through the groove 124 between the adjacent two support ribs 123, the bottom cover 32 is then rotated to support the convex ribs 321 on the corresponding support ribs 123 to install the bottom cover 32, which is convenient for assembly. In other embodiments, the bottom cover 32 may also be installed on the bottom of the casing 10 by screwing. It should be understood that the bottom cover 32 may also be fixed to the bottom of the casing 10 with screws.

In one embodiment, when the lower end of the casing 10 is provided with the reinforcement ring 122, the support rib 123 may be provided on the reinforcement ring 122 to more stably fix the support rib 123 and ensure that the support rib 123 can stably support the convex rib 321, and then fix the bottom cover 32.

In one embodiment, the support rib 123 is provided with a positioning groove 1231, and the convex rib 321 is provided with a positioning convex 322. When the convex rib 321 extends into the casing 10, the bottom cover 32 is rotated to make the positioning convex 322 on the convex rib 321 comes into the positioning groove 1231 on the support rib 123, so that the convex rib 321 and the support rib 123 are positioned to fix the bottom cover 32, which is convenient for assembly.

In one embodiment, the convex rib 321 is provided with a limit protrusion 323 for stopping a side surface of the support rib 123. During assembly, when the convex rib 321 extends into the casing 10, the bottom cover 32 is rotated, and the side of the support rib 123 blocks the limit protrusion 323, that is, the limit protrusion 323 rotates with the bottom cover 32, and when the limit protrusion 323 reaches the side of the support rib 123, the bottom cover 32 cannot continue to rotate, so as to limit the rotation of the bottom cover 32, such that a rotation position of the convex rib 321 can be determined to ensure that the convex rib 321 is well supported on the support rib 123, thereby the bottom cover 32 is installed on the casing 10. In addition, with this structure, when the bottom cover 32 needs to be removed, the bottom cover 32 can be rotated in a reverse direction the bottom cover 32 can be taken out after the limit protrusion 323 reaches a side of the adjacent support rib 123, and then the filter 30 can be taken out, which facilitates the replacement of the filter 30.

In one embodiment, the bottom surface of the bottom cover 32 is provided with two recesses 324 spaced apart, and a knob 325 is formed between the two recesses 324 to facilitate the rotation of the bottom cover 32, then the bottom cover 32 can be conveniently installed at the bottom of the casing 10. In addition, this structure also enables the bottom of the knob 325 to remain flat with the bottom of the bottom cover 32 for placement on the surface of a medium, thereby facilitating the placement of the air purifier 100.

In one embodiment, the filter 30 also includes a ring cover 33 which is installed on the upper end of the filter cylinder 31 to increase the structural strength of the filter 30 and better ensure the filter cylinder 31.

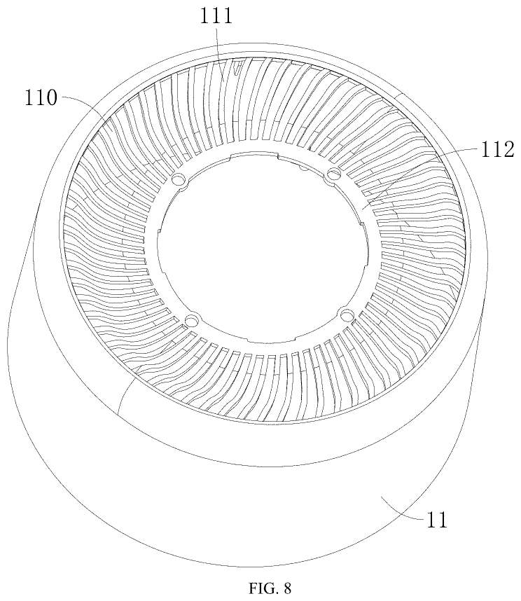

In one embodiment, referring to FIG. 2 and FIG. 7, the light source may be an LED module 41 for more energy saving.

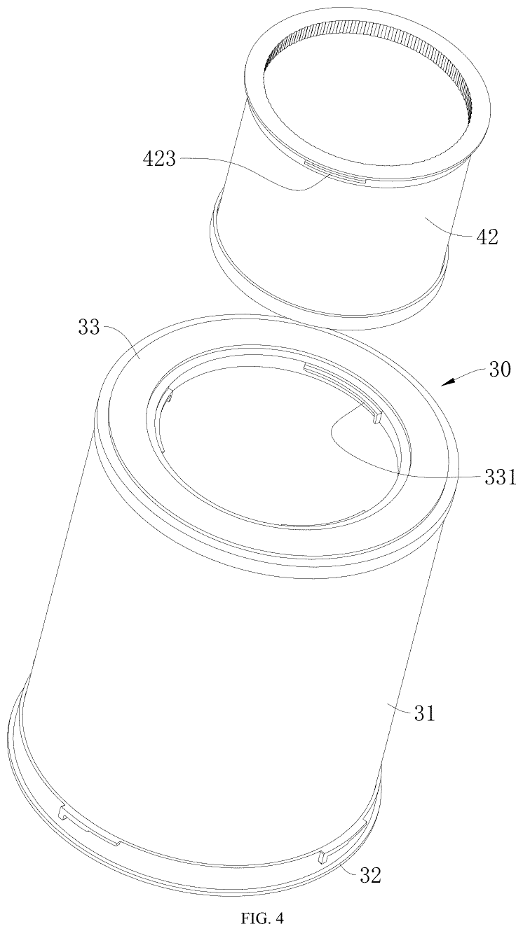

In one embodiment, referring to FIG. 4, an inner rotary buckle 331 may be provided on the ring cover 33, and an outer rotary buckle 423 may be provided on the inner filter cylinder 421. The inner filter cylinder 421 and the ring cover 33 are fixedly connected when the inner rotary buckle 331 is engaged in a snap-fitted manner with the outer rotary buckle 423. It should be understood that the upper end of the inner filter cylinder 421 may also be directly supported on the casing 10.

In some embodiments, an internal thread may be provided on the upper end of the filter cylinder 31, and an external thread may be provided on the upper end of the inner filter cylinder 421, such that the inner filter cylinder 421 and the filter cylinder 31 can be fixedly connected by matching the internal thread and external thread. This structure makes the air filtered by the filter cylinder 31 are processed by the inner filter cylinder 421, which can improve the ability and efficiency of air purification.

In one embodiment, an internal thread may be provided on the ring cover 33, and an external thread may be provided on the inner filter cylinder 421, so that the inner filter cylinder 421 and the ring cover 33 can be fixedly connected by matching the internal thread and external thread. It should be understood that the upper end of the inner filter cylinder 421 may also be directly supported on the casing 10.

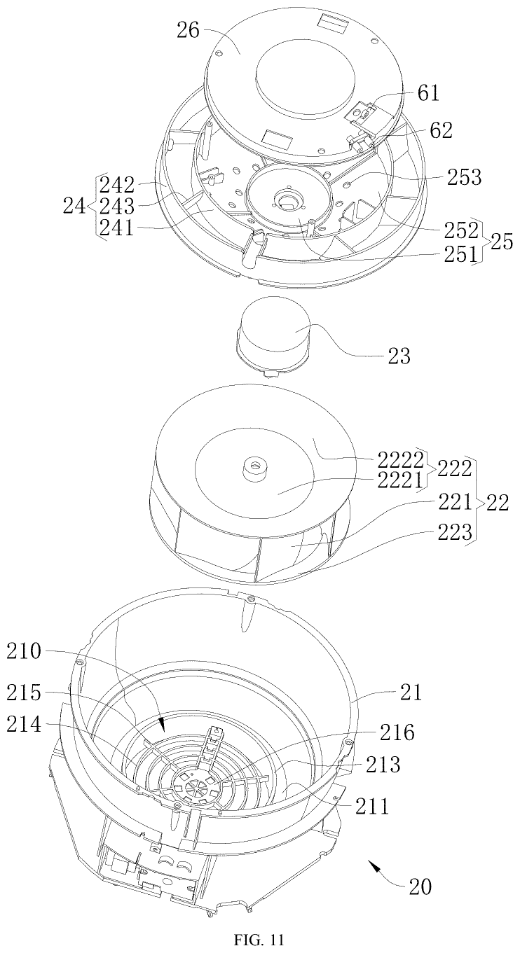

In one embodiment, referring to FIGS. 2, 10, and 11, the fan 20 includes a housing 21, a wind rotor 22, and a motor 23. The housing 21 is installed in the casing 10, and the wind rotor 22 is rotatably installed in the housing 21. Between the housing 21 and the wind rotor 22, an air duct is formed. The wind rotor 22 is connected to a motor 23, and the motor 23 drives the wind rotor 22 to rotate in the housing 21. The motor 23 is supported in the casing 10. The use of the wind rotor 22 can improve the aerodynamic performance of the fan 20 and reduce the operating noise.

In one embodiment, a ratio of the inner diameter D1 of the air outlet end of the housing 21 to the outer diameter D2 of the wind rotor 22 is in a range of 1.2-1.6, i.e., 1.2≤D1/D2≤1.6, that is, the inner diameter D1 of the air outlet end of the housing 21 is 1.2-1.6 times the outer diameter D2 of the wind rotor 22, which can not only guarantee the good aerodynamic performance of the wind rotor 22, where the wind rotor 22 can produce greater suction force at the same speed, but also can reduce the operating noise and keep the operation noise lower, so that the operating noise of the air purifier 100 can be reduced, and the purification efficiency of the air purifier 100 can be improved.

In one embodiment, the ratio of the inner diameter D1 of the air outlet end of the housing 21 to the outer diameter D2 of the wind rotor 22 is in the range of 1.3-1.5, that is, the inner diameter D1 of the air outlet end of the housing 21 is 1.3-1.5 times the outer diameter D2 of the wind rotor 22, which can better ensure the good aerodynamic performance of the wind rotor 22, and make the wind rotor 22 generate greater suction, reduce operating noise, and improve the purification efficiency of the air purifier 100.

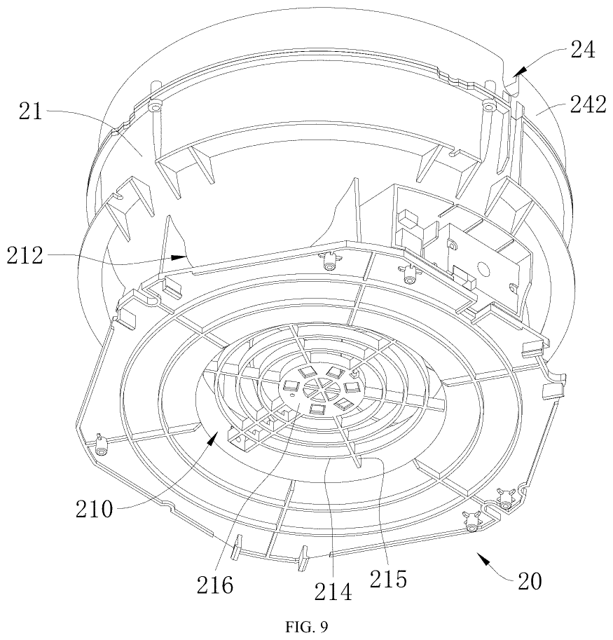

In one embodiment, referring to FIGS. 9 to 11, the wind rotor 22 includes a plurality of moving blades 221, an air intake guide ring 223, and a baffle 222. The baffle 222 is connected to the motor 23, and the moving blades 221 are connected to the air intake guide ring 223 and the baffle 222. The moving blades 221 are supported by the baffle 222. The air intake guide ring 223 is provided in connection with the moving blades 221 can increase the structural strength of the wind rotor 22 and better position and support the moving blade 221. The air intake guide ring 223 is rotatably supported in the housing 21, so that the wind rotor 22 can be positioned through the air intake guide ring 223, so as to ensure a smooth rotation of the wind rotor 22. During operation, the motor 23 drives the baffle 222 to rotate, then drives the moving blades 221 and the air intake guide ring 223 to rotate, so that air enters the moving blades 221 from the air intake guide ring 223 and flows out under pressure.

In one embodiment, referring to FIGS. 2, 10 and 11, the baffle 222 includes a flat plate portion 2221 and an inclined portion 2222. The inclined portion 2222 is arranged around the flat plate portion 2221, and the flat plate portion 2221 is connected to the inclined portion 2222 from its peripheral side. The flat plate portion 2221 is located in the middle of the baffle 222, and the inclined portion 2222 extends from the peripheral side of the flat plate portion 2221 toward a direction away from the air intake guide ring 223. With this structure, when the wind rotor 22 rotates, the inclined portion 2222 of the baffle 222 can guide the air flow to the air outlet end of the fan 20, so as to reduce aerodynamic loss and improve aerodynamic performance. It should be understood that the baffle 222 may also be provided with a curved structure with a middle part protruding toward the air intake guide ring 223. It should be noted that the baffle 222 may also be provided in a flat plate structure to facilitate processing and manufacturing.

In one embodiment, an air inlet end of the housing 21 is provided with a support plate 211, and the support plate 211 is extended inwardly from the air inlet end of the housing 21. The support plate 211 is provided with an air inlet 210, and an end of the air intake guide ring 223 away from the baffle 222 is supported on the support plate 211, that is, an inner end of the air intake guide ring 223 is supported on the support plate 211 to facilitate the support of the air intake guide ring 223, thereby ensuring a smooth rotation of the wind rotor 22. The gap between the housing 21 and the air intake guide ring 223 can be reduced, thereby reducing air backflow and improving the aerodynamic performance.

In one embodiment, a convex ring 213 is protruded on the support plate 211, and the convex ring 213 extends into the air intake guide ring 223, so that the air intake guide ring 223 can be positioned by the convex ring 213 to ensure a smooth rotation of the wind rotor 22. In addition, this structure ensures a U-shaped gap formed between the inner end of the air intake guide ring 223 and the convex ring 213 and the support plate 211, in this way, the U-shaped gap can increase the resistance of air backflow when the air flow back, thereby improving the aerodynamic performance of the fan 20.

In one embodiment, referring to FIGS. 2, 9 and 7, the inner filter cylinder 421 and the LED module 41 are supported at the air inlet 210 of the support plate 211, so that the purification module 40 is supported at the air inlet 210 of the support plate 211 to facilitate the purification of circulating air.

In one embodiment, the support plate 211 is provided with a fixing plate 216, and the LED module 41 is installed on the fixing plate 216 so as to be supported.

In one embodiment, referring to FIGS. 2, 10 and 11, the air intake guide ring 223 is bent from the outside to the inside in a direction away from the baffle 222, that is, the air intake guide ring 223 presents a C-shaped cross-section, and the inner end of the air intake guide ring 223 protrudes away from the baffle 222. In this way, the air intake guide ring 223 can better guide the air flow, reduce wind resistance, and improve aerodynamic performance. it should be understood that the air intake guide ring 223 may also be provided in a plane ring shape. In some embodiments, the air intake guide ring 223 may also be provided in a flared shape inclined to the radial direction of the air intake guide ring 223, that is, a cross-section of the air intake guide ring 223 is a plane inclined to the radial direction of the air intake guide ring 223.

In one embodiment, the housing 21 has a shrinking section 212 connected to the support plate 211, and the shrinking section 212 is arranged to shrink in a direction towards the air intake guide ring 223. In this way, the gap between the shrinking section 212 and the air intake guide ring 223 can be smaller, so as to increase the resistance of air backflow, thereby improving the aerodynamic performance of the fan 20.

In one embodiment, the shrinking section 212 has a first shrinking portion 2121 that is recessed toward the middle of the air intake guide ring 223, that is to say, the first shrinking portion 2121 is the part on the shrinking section 212 corresponding to the intake guide ring. In this way, the gap between the first shrinking portion 2121 and the air intake guide ring 223 can be smaller, and the resistance of air backflow in the fan 20 can be increased, thereby improving the aerodynamic performance of the fan 20.

In one embodiment, the shrinking section 212 has a second shrinking portion 2122 that is an end of the shrinking section 212 away from the support plate 211. The second shrinking portion 2122 shrinks inwardly, that is, the second shrinking portion 2122 is arranged to shrink in a direction towards the center position of the casing 10. In addition, the second shrinking portion 2122 is located at an end of the air intake guide ring 223 away from the support plate 211, so that the gap between the end of the air intake guide ring 223 away from the support plate 211 and the second shrinking portion 2122 can be smaller, the resistance of air backflow in the fan 20 can be increased, thereby improving the aerodynamic performance of the fan 20.

In one embodiment, as the shrinking section 212 is provided with the first shrinking portion 2121 and the second shrinking portion 2122, the gap between the shrinking section 212 and the air intake guide ring 223 is presented in an S-shape, which can further the resistance of air backflow in the fan 20 can be increased, thereby improving the aerodynamic performance of the fan 20.

In one embodiment, the end of the air intake guide ring 223 away from the support plate 211 has a convex edge 2231. The convex edge 2231 protrudes from the air intake guide ring 223 toward the support plate 211. In this way, on the one hand, the air intake guide ring 223 can be positioned through the convex edge 2231 with respect to the shrinking section 212, to ensure a smooth rotation of the wind rotor 22; on the other hand, the gap between the shrinking section 212 and the convex edge 2231 can be reduced, and the resistance of air backflow in the fan 20 can be increased, so as to improve the aerodynamic performance of the fan 20.

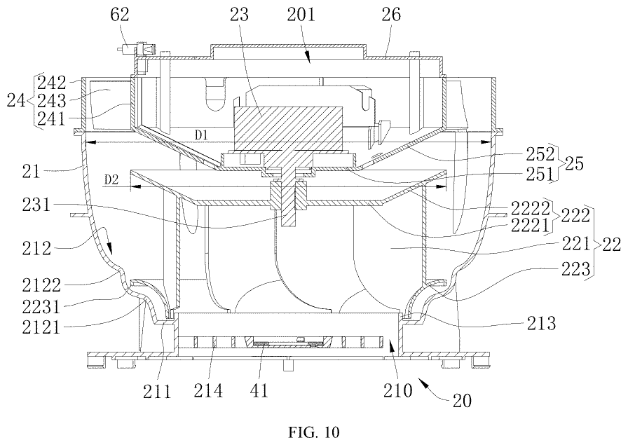

In one embodiment, referring to FIGS. 9 and 10, a plurality of air guide grids 214 and support bars 215 are disposed at the air inlet 210, and the support bars 215 is connected to these air guide grids 214 so as to support the air guide grids 214. The support bars 215 are also connected to the support plate 211. The air guide grid 214 is in a flat sheet shape, and a thickness direction of the air guide grid 214 is arranged along the radial direction of the wind rotor 22, so that the air entering the wind rotor 22 can be rectified, so as to better guide the air flow into the wind rotor 22 and improve the aerodynamic performance of the fan 20.

In one embodiment, the support bar 215 is in a sheet shape, and the support bar 215 is in the shape of a flat sheet, and the thickness direction of the support bar 215 is arranged along the radial direction of the wind rotor 22, so that the air entering the wind rotor 22 can be rectified by the support bar 215, so as to better guide the air flow into the wind rotor 22 and improve the aerodynamic performance of the fan 20.

In one embodiment, referring to FIGS. 2 and 10, the fan 20 also includes a diffuser 24, and the diffuser 24 is installed on the housing 21. The arrangement of the diffuser 24 can reduce aerodynamic losses, improve the aerodynamic performance of the fan 20, reduce an exhaust noise of the fan 20, and thereby reduce the operating noise of the fan 20.

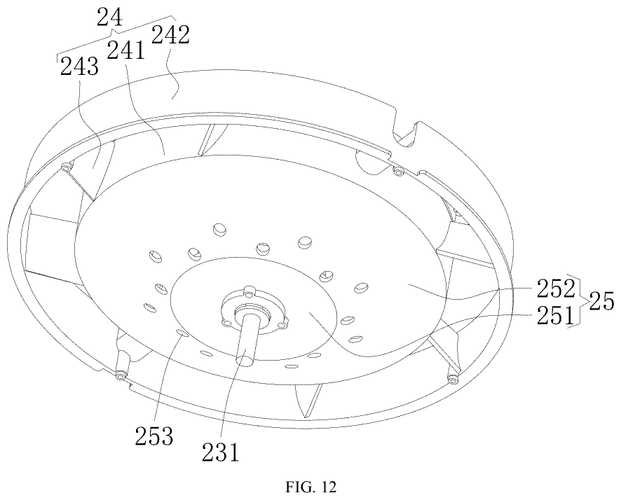

In one embodiment, referring to FIGS. 2, 10 and 12, the diffuser 24 includes an outer ring plate 242, an inner ring plate 241, and several stator blades 243. These several stator blades 243 are provided between the outer ring plate 242 and the inner ring plates 241 and connect the outer ring plate 242 and the inner ring plate 241, so that the outer ring plate 242 and the inner ring plate 241 form a passage for airflow, such that the airflow discharged from the fan 20 can be guided and rectified, which reduces the air loss, reduces the operating noise, and improves the aerodynamic performance of the fan 20. The outer ring plate 242 is connected to the housing 21 to fix the diffuser 24 on the casing 10. An outer diameter of the inner ring plate 241 is smaller than or equal to the outer diameter of the wind rotor 22 so as to prevent the inner ring plate 241 from blocking the air flow out of the fan 20 and reduce the resistance of air flow.

In one embodiment, referring to FIGS. 10 to 12, the diffuser 24 also includes a mounting plate 25 connected to the inner ring plate 241, a motor 23 is mounted on the mounting plate 25, and an output shaft 231 of the motor 23 passes through the mounting plate 25 is connected to the baffle 222 to support the motor 23 through the mounting plate 25 so as to facilitate the mounting and fixing of the motor 23. it should be understood that a separate support may also be provided to support the motor 23 in the casing 10.

In an embodiment, the diffuser 24 also includes a cover plate 26. The cover plate 26 covers an end of the inner ring plate 241 away from the mounting plate 25, so that the cover plate 26, the inner ring plate 241 and the mounting plate 25 enclose a cavity 201. The motor 23 is disposed in the cavity 201, and the output shaft 231 of the motor 23 extends out of the cavity 201 and is connected to the baffle 222, so as to better install and protect the motor 23.

In one embodiment, the mounting plate 25 includes a flat plate portion 251 and an inclined portion 252, the inclined portion 252 is arranged around the flat plate portion 251, the flat plate portion 251 is connected to the inclined portion 252 from at its peripheral side, and the flat plate portion 251 is located in the middle of the mounting plate 25, thereby facilitating the support and installation of the motor 23. The inclined portion 252 extends along the peripheral side of the flat plate portion 251 in the direction away from the wind rotor 22, and an edge of the inclined portion 252 is connected to the inner ring plate 241, so that a volume of the cavity 201 formed by the cover plate 26, the inner ring plate 241 and the mounting plate 25 can be increased to better accommodate the motor 23 and reduce the volume of the diffuser 24, thereby reducing the entire volume of the fan 20. it should be understood that the mounting plate 25 can also be arranged in a flat plate structure to facilitate processing and manufacturing.

In one embodiment, the inclined portion 252 is provided with a plurality of through holes 253 to facilitate heat dissipation

In one embodiment, referring to FIGS. 1 to 2, the air outlet 110 is provided on the top of the casing 10 so that the casing 10 is arranged in a vertical structure to reduce the occupied space.

In one embodiment, the casing 10 includes an upper casing 11 and a lower casing 12 mounted on the lower casing 12. The casing 10 is formed with the upper casing 11 and the lower casing 12, which not only facilitates processing and manufacture, but also facilitates the installation of various components in the casing 10.

In one embodiment, referring to FIGS. 1 to 2, the air purifier 100 also includes a panel 51 installed on the top of the casing 10, where the panel 51 is used to control the air purifier 100. it should be understood that, in some embodiments, buttons may also be provided on the casing 10 to control the air purifier 100.

In one embodiment, the air purifier 100 also includes a support shell 52, the support shell 52 is installed on the top of the casing 10, and the panel 51 is installed on the support shell 52, so that the panel 51 is supported on the top of the casing 10 through the support shell 52. The panel 51 and the casing 10 are arranged with a gap so that air can flow out between the casing 10 and the panel 51.

In one embodiment, referring to FIGS. 1, 2 and 8, the top of the casing 10 is provided with a plurality of ribs 111 and connection rings 112. These ribs 111 are arranged around the middle of the casing 10, and an inner end of each rib 111 is connected to the connection ring 112, that is, the end of each rib 111 close to the middle of the casing 10 is connected to the connection ring 112, so as to ensure a good structural strength on the top of the casing 10. An air outlet hole 110 is formed between two adjacent ribs 111. This structure can form a circle of air outlets 110 on the top of the casing 10 to facilitate the spread of the purified air flow to the surroundings.

In one embodiment, the support shell 52 may be connected to the connection ring 112 to support the panel 51 on the top of the casing 10. It is understood that the panel 51 may also be installed on and supported by the connection ring 112.

In one embodiment, the support shell 52 is horn-shaped, and the diameter of a lower end of the support shell 52 is smaller than the diameter of an upper end of the support shell 52, so that when the purified air flows out from the air outlet 110, the support shell 52 can guide the air to diffuse around the peripheral side of the casing 10 to increase the area covered by the purified air.

In one embodiment, referring to FIGS. 2 and 8, the air purifier 100 also includes an air guide shell 53, and the air guide shell 53 is provided in the casing 10 with one end being connected to the inner ring plate 241, and the other end being connected to the connection ring 112. The air guide shell 53 is provided to better support the connection ring 112 and guide the airflow to the air outlet 110.

In one embodiment, the air guide shell 53 includes an annular section 531 and a contraction section 532. The annular section 531 is arranged along the axial direction of the wind rotor 22. One end of the annular section 531 is connected to the contraction section 532, and the other end of the annular section 531 is connected to the inner ring plate 241. The outer diameter of the connection ring 112 is smaller than the inner diameter of the inner ring plate 241, and the contraction section 532 extends from the end of the annular section 531 away from the inner ring plate 241 toward the connection ring 112, so that the space between the air guide shell 53 and the casing 10 gradually increases from the annular section 531 to the air outlet 110, so that the pressure can be better diffused, the airflow loss can be reduced, and the aerodynamic performance of the fan 20 and the purification efficiency of the air purifier 100 can be improved.

In one embodiment, the air guide shell 53 also includes a support plate 533. An edge of the support plate 533 is connected to the contraction section 532. The support plate 533 covers the connection ring 112, so as to better support the connection ring 112 and ensure good structural strength of the air guide shell 53.

In one embodiment, referring to FIGS. 2, 10 and 11, the air purifier 100 also includes a negative ion generator 61 and a negative ion emission needle 62. The negative ion emission needle is electrically connected to the negative ion generator 61. The negative ion generator 61 is installed in the casing 10, and the negative ion emission needle is arranged at the air outlet 110. In this way, the air purifier 100 can generate negative ions for sterilization, thereby improving the efficiency and quality of air purification.

In one embodiment, the negative ion generator 61 is installed in the cavity 201 to ensure the negative ion generator 61. The negative ion emission needle is installed on the cover plate 26, then the air flow discharged by the fan 20 passes through the negative ion emission needle, so that the discharged air flow contains negative ions.

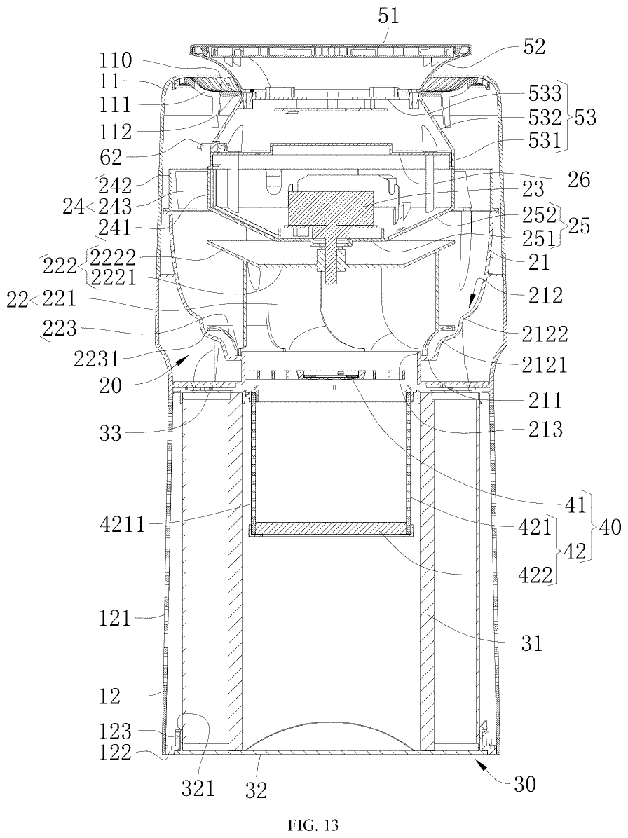

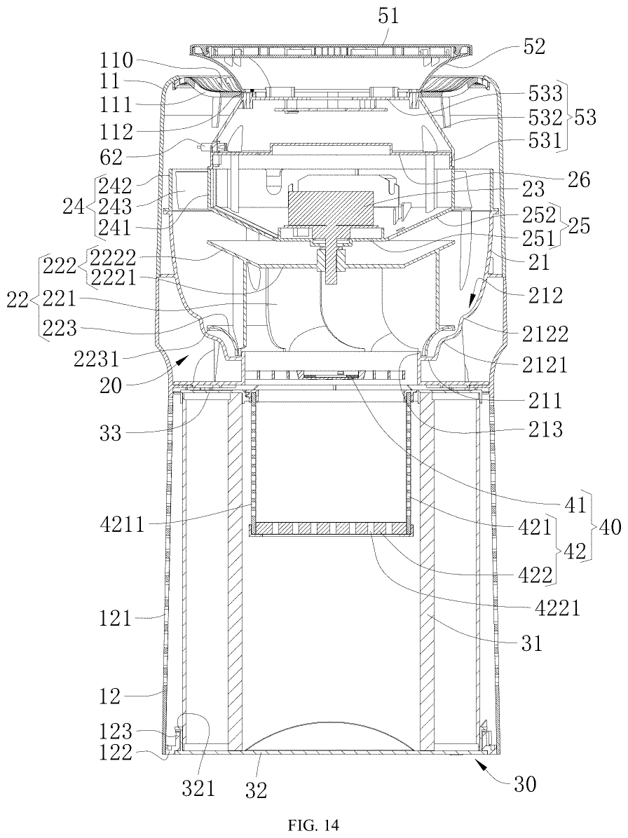

Referring to FIG. 13, it is shown a schematic cross-sectional structure diagram of the air purifier 100 provided by the embodiment. The air purifier 100 in this embodiment is a modification on the basis of the air purifier shown in FIG. 2. The air purifier 100 of this embodiment differs from the air purifier shown in FIG. 2 in that:

The filter 30 is in a cylindrical shape. The purification module 40 includes an inner filter 42, which includes the inner filter cylinder 421 and the purification plate 422. The inner filter cylinder 421 is cylindrical, and the purification plate 422 is installed at a bottom of the inner filter cylinder 421. The inner filter cylinder 421 is configured to remove harmful gases in the air, such as formaldehyde, benzene, TVOC and the like. The inner filter cylinder 421 is disposed in the filter 30 to reduce the occupied space and improve the space utilization rate. The inner filter cylinder 421 is supported in the casing 10, so that the inner filter 42 is supported in the casing 10. The purification plate 422 can also play a role in purifying the air to improve the efficiency of air purification.

The inner filter cylinder 421 is a honeycomb-like cylinder or a reticulate cylinder. The inner filter cylinder 421 is a honeycomb-like cylinder, that is, the inner filter cylinder 421 is cylindrical as a whole, and a plurality of honeycomb-like vents 4211 are disposed on the side wall of the inner filter cylinder 421. The inner filter cylinder 421 of the honeycomb-like cylinder has low resistance to air flow, and allows more air flow to pass through the purification plate to improve the ability to remove formaldehyde, benzene, TVOC and other harmful gases. The purification plate 422 can also play a role in filtering the air to improve the air purification capability. The inner filter cylinder 421 is a reticulate cylinder, that is to say, the inner filter cylinder 421 is cylindrical as a whole, and a plurality of vents 4211 are disposed on the side wall of the inner filter cylinder 421 to make the inner filter cylinder 421 a reticulate cylinder. The inner filter cylinder 421 is reticulate which provides low resistance to air flow, and allows more air flow to pass through the purification plate to improve the ability to remove formaldehyde, benzene, TVOC and other harmful gases.

The air purifier 100 of this embodiment is provided with an inner filter cylinder 421 that can remove harmful gases in the air to improve the purification capability. The inner filter cylinder 421 is a honeycomb-like cylinder or a reticulate cylinder so as to reduce wind resistance of the inner filter cylinder 421 and reduce air resistance, thereby reducing the speed and noise of the fan 20. The filter 30 is cylindrical, and the inner filter cylinder 421 is disposed in the filter 30, which can reduce the occupied space, and the air filtered by the filter 30 can better enter the inner filter 42 for filtration, and then enter the fan 20, so that the purification efficiency is higher.

In one embodiment, the inner filter cylinder 421 may be made of a substrate, for example, the substrate is bent into a cylindrical shape to form the inner filter cylinder 421. The substrate is honeycomb-like or reticulate, then the inner filter cylinder 421 manufactured with this substrate is honeycomb-like or reticulate.

In one embodiment, the substrate may be made of activated carbon corrugated paper to form a reticulate structure so that harmful gases such as formaldehyde, benzene, TVOC and the like in the air can be removed.

In one embodiment, the substrate may be made of a honeycomb-like activated carbon to form a honeycomb-like or reticulate structure, so as to remove harmful gases such as formaldehyde, benzene, TVOC, etc. in the air.

In one embodiment, the substrate may be made of a honeycomb-like carbon-filled filter screen, that is, a honeycomb-like support plate is disposed between two layers of filter screens, and activated carbon particles are disposed in the holes of the honeycomb-like support plate to form a honeycomb-like or reticulate structure, so as to remove harmful gases such as formaldehyde, benzene, TVOC, etc. in the air.

In one embodiment, the substrate may be made of a honeycomb-like or reticulate substrate with a coating being sprayed on the surface that removes harmful gases in the air. The coating may be a manganese compound layer, a photocatalyst layer or a cold catalyst layer, etc., so that the substrate can remove harmful gases such as formaldehyde, benzene, and TVOC in the air.

In one embodiment, the purification plate 422 is a photocatalytic filter, that is to say, the purification plate 422 is made of a photocatalytic net, or the purification plate 422 is provided with a photocatalytic net. The purification module 40 also includes a light source, which is installed in and supported by the casing 10. The light source is used to irradiate the purification plate 422, so that the purification plate 422 can decompose organic molecules in the passing air, such as harmful molecules of formaldehyde, benzene, TVOC, so as to play a role in purification and sterilization, which improves purification capability. It should be understood that the purification plate 422 may also use a high-efficiency filter layer to improve the air purification capability.

In one embodiment, the purification module 40 also includes a light source, which may be an ultraviolet light source. The light source is installed in the casing 10 so as to be supported by the casing 10. The light source emits ultraviolet light to the inner filter cylinder 421 so as to perform sterilization and disinfection.

Referring to FIG. 14, it is shown a schematic cross-sectional structure diagram of the air purifier 100 provided by the embodiment. The air purifier 100 in this embodiment is a modification on the basis of the air purifier shown in FIG. 2. The air purifier 100 of this embodiment differs from the air purifier shown in FIG. 2 in that:

The inner filter cylinder 421 is a honeycomb-like cylinder or a reticulate cylinder. In addition, the purification plate 422 is honeycomb-like or reticulate.

The inner filter cylinder 421 is a honeycomb-like cylinder, that is, the inner filter cylinder 421 is a cylinder as a whole, and a plurality of honeycomb-like vents 4211 are disposed on the side wall of the inner filter cylinder 421. The inner filter cylinder 421 is a honeycomb-like cylinder provides low resistance to air flow, and allows more air flow to pass through the purification plate to improve the ability to remove formaldehyde, benzene, TVOC and other harmful gases. The purification plate 422 can also play a role in filtering the air to improve the air purification capability. The inner filter cylinder 421 is a reticulate cylinder, that is to say, the inner filter cylinder 421 is cylindrical as a whole, and a plurality of vents 4211 are disposed on the side wall of the inner filter cylinder 421 to make the inner filter cylinder 421 a reticulate cylinder. The inner filter cylinder 421 of the reticulate cylinder has low resistance to air flow, and allows more air flow to pass through the purification plate to improve the ability to remove formaldehyde, benzene, TVOC and other harmful gases.

The purification plate 422 is honeycomb-like, that is, the purification plate 422 is provided with a plurality of honeycomb-like openings 4221. The use of the honeycomb-like purification plate 422 has low resistance to airflow, and more airflow can pass through the purification plate to improve the ability to remove formaldehyde, benzene, TVOC and other harmful gases. The inner filter cylinder 421 can also play a role in filtering air, improving the air purification capability. The purification plate 422 is reticulate, that is, the purification plate 422 is provided with a plurality of openings 4221 so that the purification plate 422 is reticulate. The use of the reticulate purification plate 422 has low resistance to airflow, and more airflow can pass through the purification plate to improve the ability to remove formaldehyde, benzene, TVOC and other harmful gases.

In one embodiment, the purification plate 422 is disposed at the bottom of the inner filter cylinder 421 to facilitate assembly. In other embodiments, the purification plate 422 may also be provided inside the inner filter cylinder 421, that is to say, the inner filter cylinder 421 is arranged around the purification plate 422. For example, the purification plate 422 may be arranged in the middle or upper part of the inner filter cylinder 421 in a height direction.

The air purifier 100 of the embodiment of the present application has high air purification efficiency, low operating noise, long service life, convenient replacement of the filter 30, wide coverage area of purified air, and compact structure.

The above are only optional embodiments of the present application, which are not intended to limit the present application. Any modification, equivalent replacement and improvement made within the spirit and principle of this application shall be included in the protection scope of the present application.

What is claimed is:

1. An air purifier, comprising:
a casing, a fan and a filter, wherein the fan and the filter are installed in the casing, and the filter is in a cylindrical shape; wherein the casing defines air intake holes at a position in the proximity of the filter, and defines an air outlet at an end of the casing in the proximity of an air outlet end of the fan; and
a purification module comprising an inner filter for purification of harmful gases in air, and the inner filter comprising:
an inner filter cylinder having a cylindrical shape; and
a purification plate installed in the inner filter cylinder and configured for the purification of harmful gases in the air, wherein the purification plate is honeycomb-like or reticulate, the inner filter cylinder is disposed in the filter, and an upper end of the inner filter cylinder is supported in the casing.

2. The air purifier according to claim 1, wherein the upper end of the inner filter cylinder is adaptively connected to an upper end of the filter.

3. The air purifier according to claim 2, wherein the upper end of the filter is provided with an internal thread, the upper end of the inner filter cylinder is provided with an external thread, and the internal thread is engaged with the external thread in a fitted manner; or alternatively,
the upper end of the filter is provided with an inner rotary buckle, the upper end of the inner filter cylinder is provided with an outer rotary buckle, and the inner rotary buckle is engaged in a snap-fitted manner with the outer rotary buckle.

4. The air purifier according to claim 1, wherein the inner filter cylinder is a photocatalytic filter, and the purification module further comprises a light source installed in the casing and configured to irradiate the inner filter cylinder.

5. The air purifier according to claim 1, wherein the purification module further comprises a light source installed in the casing and configured for emitting ultraviolet light toward the inner filter cylinder.

6. The air purifier according to claim 1, wherein the purification plate is made of a honeycomb-like or reticulate substrate; and
the substrate is an activated carbon cardboard; or
the substrate is a honeycomb-like carbon-filled filter; or
the substrate has a surface being sprayed with a coating that removes harmful gases in the air, wherein the coating is a manganese compound layer, a photocatalyst layer or a cold catalyst layer.

7. The air purifier according to claim 1, wherein the inner filter cylinder is a honeycomb-like cylinder or a reticulate cylinder.

8. The air purifier according to claim 7, wherein the inner filter cylinder is made of a honeycomb-like or reticulate substrate; and
the substrate is an activated carbon cardboard; or
the substrate is a honeycomb-like carbon-filled filter; or
the substrate has a surface being sprayed with a coating that removes harmful gases in the air, wherein the coating is a manganese compound layer, a photocatalyst layer or a cold catalyst layer.

9. An air purifier comprising:
a casing, a fan and a filter, wherein the fan and the filter are installed in the casing, and the filter is in a cylindrical shape; wherein the casing defines air intake holes at a position in the proximity of the filter, and defines an air outlet at an end of the casing in the proximity of an air outlet end of the fan; and
a purification module comprising an inner filter for purification of harmful gases in air, the inner filter comprising:
an inner filter cylinder for the purification of harmful gases in the air; and
a purification plate installed at a bottom of the inner filter cylinder, and configured to purify the air, wherein the purification plate is a honeycomb-like cylinder or a reticulate cylinder, wherein the inner filter cylinder is disposed in the filter, and an upper end of the inner filter cylinder is supported in the casing.

10. The air purifier according to claim 9, wherein the inner filter cylinder is made of a honeycomb-like or reticulate substrate; wherein the substrate is an activated carbon cardboard; or the substrate is a honeycomb-like carbon-filled filter; or the substrate has a surface being sprayed with a coating that removes harmful gases in the air, and the coating is a manganese compound layer, a photocatalyst layer or a cold catalyst layer.

11. The air purifier according to claim 2, wherein the inner filter cylinder is a photocatalytic filter, and the purification module further comprises a light source installed in the casing and configured to irradiate the inner filter cylinder.

12. The air purifier according to claim 2, wherein the purification module further comprises a light source installed in the casing and configured for emitting ultraviolet light toward the inner filter cylinder.

13. The air purifier according to claim 2, wherein the purification plate is made of a honeycomb-like or reticulate substrate; and
the substrate is an activated carbon cardboard; or
the substrate is a honeycomb-like carbon-filled filter; or
the substrate has a surface being sprayed with a coating that removes harmful gases in the air, wherein the coating is a manganese compound layer, a photocatalyst layer or a cold catalyst layer.

14. The air purifier according to claim 2, wherein the inner filter cylinder is a honeycomb-like cylinder or a reticulate cylinder.

15. The air purifier according to claim 14, wherein the inner filter cylinder is made of a honeycomb-like or reticulate substrate; and
the substrate is an activated carbon cardboard; or
the substrate is a honeycomb-like carbon-filled filter; or
the substrate has a surface being sprayed with a coating that removes harmful gases in the air, wherein the coating is a manganese compound layer, a photocatalyst layer or a cold catalyst layer.

16. The air purifier according to claim 3, wherein the inner filter cylinder is a photocatalytic filter, and the purification module further comprises a light source installed in the casing and configured to irradiate the inner filter cylinder.

17. The air purifier according to claim 3, wherein the purification module further comprises a light source installed in the casing and configured for emitting ultraviolet light toward the inner filter cylinder.

18. The air purifier according to claim 3, wherein the purification plate is made of a honeycomb-like or reticulate substrate; and
the substrate is an activated carbon cardboard; or
the substrate is a honeycomb-like carbon-filled filter; or
the substrate has a surface being sprayed with a coating that removes harmful gases in the air, wherein the coating is a manganese compound layer, a photocatalyst layer or a cold catalyst layer.

19. The air purifier according to claim 3, wherein the inner filter cylinder is a honeycomb-like cylinder or a reticulate cylinder 20. The air purifier according to claim 19, wherein the inner filter cylinder is made of a honeycomb-like or reticulate substrate; and
the substrate is an activated carbon cardboard; or
the substrate is a honeycomb-like carbon-filled filter; or
the substrate has a surface being sprayed with a coating that removes harmful gases in the air, wherein the coating is a manganese compound layer, a photocatalyst layer or a cold catalyst layer.
